(19) United States
(12) Patent Application Publication
(10) Pub. No.: US 2008/0039485 A1
(43) Pub. Date: Feb. 14, 2008

(54) LOW-DOSE-LONG-TERM PHARMACEUTICAL COMPOSITION COMPRISING CAMPTOTHECIN DERIVATIVES FOR THE TREATMENT OF CANCERS

(75) Inventors: Li-Kang Ho, Taipei (TW); Yueh-Hsin Ping, Taipei (TW); Hsin-Chen Lee, Taipei (TW); Jane-Jen Wang, Taipei (TW); Ming-Fong Lu, Taipei (TW)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: TARGETGEN BIOTECHNOLOGY CO., LTD., Taipei (TW)

(21) Appl. No.: 11/463,099

(22) Filed: Aug. 8, 2006

(51) Int. Cl. A61K 31/4745

(52) U.S. Cl. 514/283

(57) ABSTRACT

An oral pharmaceutical composition for the treatment of digestive tract cancers contains an effective amount of a camptothecin derivative dissolved in a non-aqueous solvent. The camptothecin derivative is administered at a dose of about 0.05 to about 1 mg/kg body weight/day. The pharmaceutical composition is particularly suitable for use in low-dose-long-term regimens for colorectal cancers.

### FIELD OF THE INVENTION

The present invention relates generally to an oral pharmaceutical composition comprising a camptothecin derivative for the treatment of digestive tract cancers.

### BACKGROUND OF THE INVENTION

Camptothecin (CPT), a natural alkaloid produced by the plant Comptotheca acuminate, is an active component responsible for the anticancer activity of the extract of Comptotheca acuminate. CPT was found to be a TOP1-targeting anticancer drug, which showed highly S phase-specific cytotoxicity and induced G2-M cell cycle arrest, and it exhibits a board spectrum antitumor activity against a panel of solid tumors in animal models. Previous studies show that a replication fork collision model has been proposed to explain the S phase cytotoxicity and it is presumably important for the antitumor activity of CPT.

Several CPT derivatives have been developed recently into the clinic, such as CPT-11 (also known as irinotecan) and topotecan. Among them, 10-hydroxy-camptothecin (10-HCPT) has a wide spectrum of anticancer activity both in vitro and in vivo, and plays the same significant role in TOP1-targeting as CPT. 10-HCPT has an inhibitory effect on phosphorylation of histone H1 and H3 in murine hepatoma cells (Ling, Y. H. and Xu, B., Acta Pharmacol Sinica 14: 546 (1993)), and differentiation inducing effect in human Hep G2 cells (Zhang, X. et al., Chin Med J 113 (8): 712-3 (2000)). In addition, 10-HCPT is a promising anticancer agent has undergone clinical trials with less toxic and more active effect than other clinically approved CPT derivatives (Han, R., China Stem Cells 12: 53 (1994)).

Colorectal cancer (CRC) is a major cause of mortality in the Western world. For many years, standard chemotherapy for advanced CRC was based on the thymidylate-synthase inhibitor 5-FU. Although chemotherapy and radiation therapy have been attempted in either adjuvant or palliative treatments, nearly half of patients with CRC still die of metastatic diseases after curative surgery.

Currently CPT derivatives have become drugs mainly used for CRC treatment. Although CPT derivatives have been shown to be highly effective in the treatment of CRC, the dosage is limited by toxicities such as diarrhea. In this regard, drug development is needed to improve chemotherapy of patients with locally advanced or metastatic colon carcinoma. The current drug modification is also essential and important to progress. For example, low-dose-long-term strategy was reconsidered for cancer treatment recently. Besides, the way of oral administration in the clinic appears to be ideal for chemotherapeutic patients. In view of the above, developing new therapeutic drugs and more effective adjuvant therapy for CRC is a worthwhile task.

### BRIEF SUMMARY OF THE INVENTION

The present invention is directed to a pharmaceutical composition that obviates one or more problems resulting from the limitations and disadvantages of the prior art.

In accordance with one embodiment of the present invention, there is provided an oral pharmaceutical composition for the treatment of digestive tract cancers comprising an effective amount of a camptothecin derivative dissolved in a non-aqueous solvent, characterized in that the camptothecin derivative is administered at a dose of about 0.05 to about 1 mg/kg body weight/day.

Also in accordance with the present invention, there is provided a method for treating digestive tract cancers in a subject comprising orally administering to the subject a pharmaceutical composition comprising an effective amount of a camptothecin derivative dissolved in a non-aqueous solvent at a dose of about 0.05 to about 1 mg/kg body weight/day of the camptothecin derivative.

Additional features and advantages of the present invention will be set forth in part in the description which follows, and in part will be obvious from the description, or may be learned by practice of the invention. The features and advantages of the invention will be realized and attained by means of the elements and combinations particularly pointed out in the appended claims.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIGS. 1A-1D show the effect of 10-HCPT and CPT-11 on cell growth of human colon cancer HT-29 and Colo-205 cells; wherein cells were treated with various concentrations of 10-HCPT or CPT-11 for 1-3 days, and the relative number of the viable cells was determined by MTT assay;

FIG. 2 shows the effect of 10-HCPT on cell growth of human colon cancer Colo-205 cells; wherein cells were treated with 5 to 20 nM of 10-HCPT for different time periods as shown in x-axis. The viable cell number was directly counted by trypan blue exclusion assay;

FIGS. 3A-3E show the effect of 10-HCPT on cell cycle distribution of Colo-205 cells. Cells were treated with various concentrations (as indicated above each of panels A to E) of 10-HCPT for 48 hrs; wherein the cell cycle distribution of these cells was analyzed by propidium iodide staining and flow cytometry;

FIG. 4 shows the results of western blotting analysis of the cell cycle regulatory proteins; wherein Colo-205 cells were treated with 3 μM of 10-HCPT for 24 hrs and assessed the expression of regulatory proteins of cell cycle as well as apoptosis by western blotting assay;

FIGS. 5A and 5B show that 10-HCPT can induce apoptosis in Colo-205 cell line: (A) DNA fragmentation assay; (B) Evaluation of DEVD-dependent cleavage activity by caspase-3; and

FIGS. 6A and 6B shows the in vivo antiproliferation effect of 10-HCPT for Colo-205 xenografts.

### DETAILED DESCRIPTION OF THE INVENTION

It is found in the invention that camptothecin derivatives are unexpectedly effective in the treatment of digestive tract cancers, particularly CRC, when dissolved in an aqueous solvent and administered orally. In this way, camptothecin derivatives become suitable for use in a low-dose-long-term regimen, which is more beneficial to cancer patients due to lower toxicities.

As used herein, the term “camptothecin derivative” comprises camptothecin per se as well as analogs derived from camptothecin, such as the known compounds 10-HCPT, CPT-11, topotecan, rubitecan, lurtotecan and exatecan (see Thomas, C. J. et al., Bioorg Med Chem 12: 1585-604 (2004); and Lorence, A. and Nessler, C. L., Phytochem 65: 2735-49 (2004)). Preferably, the term “camptothecin derivative” refers to a compound of the following formula:

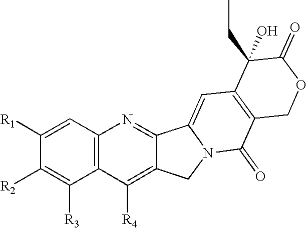

wherein R₁ represents H, —OCH₂O or —O(CH₂)₂O; R₂ represents H, OH, —OCH₂O, —O(CH₂)₂O, —OR wherein R is —C(O)—Cₙ alkyl and n is 9, 11, 13, 15, 17 or 19, or —OC(O)-piperidyl-piperidyl; R₃ represents H or —CH₂N(CH₃)₂; and R₄ represents H, methyl, ethyl, n-butyl, —Si(CH₃)₃ or —Si(CH₃)₂-t-butyl.

As used herein, the term “liquid vehicle” refers to a pharmaceutically acceptable liquid solvent able to dissolve or suspend CPT derivatives. Suitable non-aqueous solvents include, but are not limited to propylene glycol, DMSO, PEG and cremophor. It is well known in the art that CPT is water-insoluble. Some CPT derivatives, like topotecan and irinotecan, have better water-solubility than CPT, but their water-solubility is still limited. Therefore, if an aqueous vehicle is desired, it is first necessary to dissolve the derivative in a non-aqueous solubilizing agent, such as one of the above solvents, for use in the aqueous vehicle.

As used herein, the term “digestive tract cancer” refers to cancers in the digestive tract and digestion-related organs, such as esophageal cancer, stomach cancer, gastrointestinal cancer, gallbladder and bile duct cancer, liver cancer, pancreatic cancer, and colorectal cancer.

According to the present invention, the dose of the composition to be administered can vary in accordance with the age, size, and condition of the subject to be treated. Useful dosages of the composition are those which will deliver about 0.05 to about 1 mg/kg body weight/day, and preferably deliver about 0.15 to about 0.5 mg/kg body weight/day of the camptothecin derivative. The amount of the camptothecin derivative needed in the composition should be an amount suitable for oral administration in view of the metabolism of the compound and of the composition.

The pharmaceutical composition of the present invention can be manufactured by conventionally known methods with one or more excipients that are normally employed in oral liquid formulations, such as surfactants, solubilizers, stabilizers, emulsifiers, thickeners, coloring agents, sweetening agents, flavoring agents, and preservatives. Such excipients are known to those skilled in the art.

The pharmaceutical composition of the present invention can be prepared as any liquid formulation suitable for oral administration, such as syrups, elixirs, oral drops, and oral sprays.

The pharmaceutical composition of the present invention may be used in combination with other conventional cancer treatments, such as surgery, radiotherapy and biological therapy. The pharmaceutical composition of the present invention may also be used in combination with other anticancer drugs in chemotherapy.

The effect of the present invention can be well understood from the following experimental examples, which are intended to be a way of illustrating but not limiting the present invention.

### EXAMPLE 1

### Effects of 10-HCPT and CPT-11 in Inhibiting Cell Growth of Human Colon Cancer Cell Lines Colo-205 and HT-29

Two human colon cancer cell lines, Colo-205 (ATCC accession No. CCL-222) and HT-29 (ATCC accession No. HTB-38), were used in this study. Cells were treated with 0.2, 0.76, 1.52, 7.62, 15.24, 22.87, 30.49, 38.11, 45.73 or 53.35 μM 10-HCPT or CPT-11 for 1-3 days, and the relative number of the viable cells was determined by MTT assay as described below.

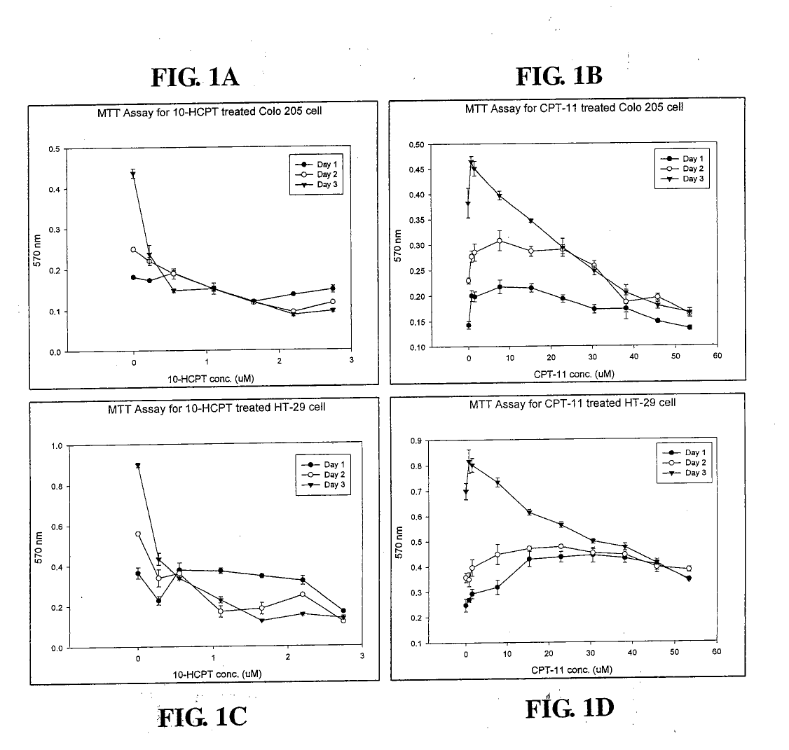

Cells were seeded in a 24-well cell culture cluster (Costar, Cambridge, Mass.) at a density of 1×10⁶ cells per mL and cultured overnight prior to drug treatment. After 10-HCPT or CPT-11 treatment for 3 days, the medium was discarded and replaced with an equal volume (0.5 mL) of fresh medium containing 0.456 mg/mL 3-[4,5-dimethylthiazol-2-yl]-2,5-diphenyl-tetrazolium bromide (MTT; Sigma Chemical Company) and incubated for 2 hours at 37° C. in the dark. The medium was discarded, and cells were then combined with 300 mL dimethyl sulfoxide (Sigma Chemical Company) to dissolve the formazan produced. Cell viability was determined according to the calorimetric comparison by reading optical density (OD) values from a microplate reader (Spectra Max 250; Spectra Diode Laboratories, CA) at an absorption wavelength of 570 nm. The results are shown in FIGS. 1A-1D.

As shown in FIGS. 1A-1D, we observed an inhibition of cell growth in 10-HCPT or CPT-11-treated cells as compared with vehicle controls. Significant growth inhibition was observed on day 2 and day 3 in Colo-205 cells after treatment with 0.2 or 22.87 μM 10-HCPT or CPT-11, respectively. Complete growth inhibition was observed on days 1-3 in Colo-205 and HT-29 cells after treatment with 1 or 0.5 μM 10-HCPT, respectively. In contrast, complete growth inhibition was observed after treatment with 38.11 and 45.73 μM CPT-11 on days 1-3 in Colo-205 and HT-29 cells, respectively.

### EXAMPLE 2

### Effect of 10-HCPT in Inhibiting Cell Growth and Reducing Cell Viability of Human Colon Cancer Cell Line Colo-205

To investigate the effect of 10-HCPT on the growth of Colo-205 cell line, the cells were treated with a series of dosages of 10-HCPT and harvested at different time points. The cell numbers were determined by trypan blue exclusion assay as described below.

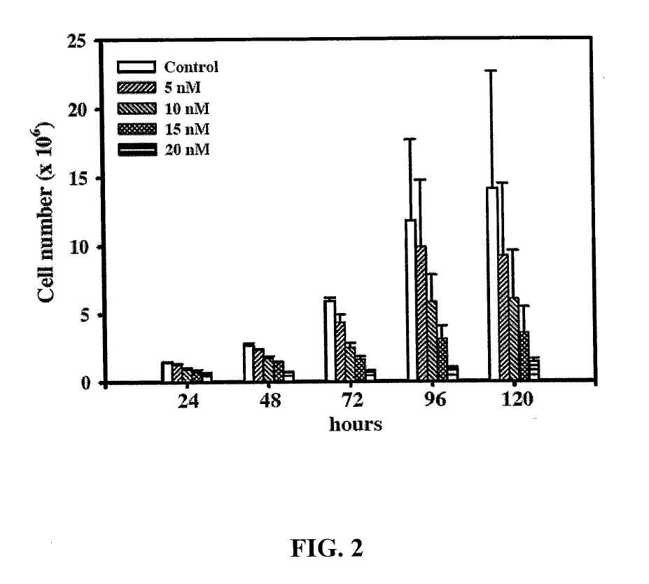

Colo-205 cells (5×10⁵) were seeded in 25T flasks overnight and then treated without (control) and with 5, 10, 15, or 20 nM of 10-HCPT, respectively. After treatment for 24 to 120 hr, cells were harvested by trypsin-EDTA and then centrifuged at 1500 rpm for 5 min at 4° C. The cell pellet was resuspended in culture medium containing 0.04% trypan blue and the viable cells were enumerated by a hemocytometer. The results are shown in FIG. 2.

As can be seen from FIG. 2, compared to the untreated cells whose cell number was increased with a doubling time of 48 hrs, the 10-HCPT treated cells did show clearly the suppression of cell number increasing in dose-dependent manner. When Colo-205 cells were treated with 20 nM of 10-HCPT, the cell number detected at 120 hrs was similar with the one measured at the first 24 hrs. This result indicates that 10-HCPT suppresses cell growth.

### EXAMPLE 3

### Effect of 10-HCPT in Disturbing Cell Cycle Distribution of Human Colon Cancer Cell Line Colo-205

We further investigated if the suppression of cell growth induced by 10-HCPT on Colo-205 was due to either cell cycle arrest or cell death. The Colo-205 cells were treated with various concentrations of 10-HCPT for 48 hrs and analyzed by propidium iodide staining and flow cytometry as described below.

After treatment with 10-HCPT, cells were trypsinized and resuspended in 70% ethanol. The cells were incubated on ice for at least 1 hr and resuspended in 1 mL of cell cycle assay buffer (0.38 mM sodium citrate, 0.5 mg/mL RNase A, and 0.01 mg/mL propidium iodide) at a concentration of 5×105 cells/mL. Samples were stored in the dark at 4° C. until cell cycle analysis, which was carried out by use of a flow cytometer and ModFit LT 2.0 software (Verity Software, Topsham, Me.).

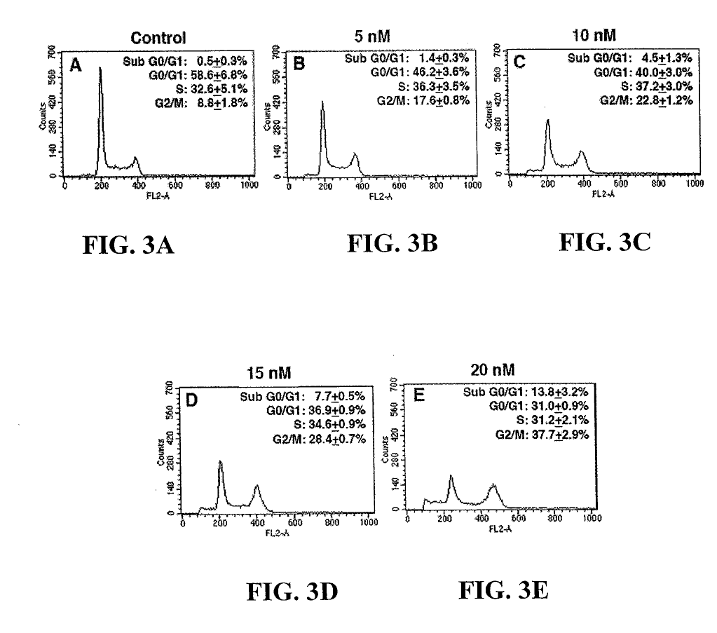

A FACScan flow cytometer (Becton Dickinson, Bedford, Mass.) equipped with a 488-nm argon laser was used for analysis of cellular function by flow cytometry. Forward and side scatters were used to establish size gates and exclude cellular debris from the analysis. The excitation wavelength was set at 488 nm. The observation wavelength of 530 nm was chosen for green fluorescence and 585 nm for red fluorescence and the intensities of emitted fluorescence were collected on FL1 and FL2 channels, respectively. In each measurement, a minimum of 20,000 cells was analyzed. Data were acquired and analyzed using the Cell Quest software (Becton Dickinson). Relative change in the mean fluorescence intensity was calculated as the ratio between mean fluorescence intensity in the channel of the treated cells and that of the control. The results are shown in FIGS. 3A-3E.

As shown in FIGS. 3A-3E, no significant differences were seen in S phase of cell cycle between untreated and 10-HCPT treated cells. The treatment of 10-HCPT, however, induced dramatically the increasing of the cell populations in G2/M phase and the decreasing of the ones in G0/G1 phase. It implied that 10-HCPT might cause cell cycle arrest on Colo-205. In addition, the flow cytometric analysis also revealed that the increasing population of Colo-205 in sub G0/G1 phase was induced by 10-HCPT. The accumulation of cell numbers in both sub G0/G1 and G2/M phases were shown in 10-HCPT-treated dose-dependent manner (FIGS. 3F and G). Taken together, these results suggested that the 10-HCPT induced suppression of cell growth might result from cell cycle arrest as well as apoptosis.

### EXAMPLE 4

### Effect of 10-HCPT in Inducing Cell Cycle Arrest in G2/M Phase

The progression of the cell cycle is promoted by various CDKs binding with their respective cyclin partners and inhibited by CDK inhibitors such as p21 and p27. In general, CDK2 in association with cyclin E regulates the G1/S transition, and CDK1 in concert with type-A and -B cyclins controls the G2/M transition. Both cell cycle inhibitory proteins p21 and p27 block cell cycle progression at the G1/S as well as the G2/M transitions of cell cycle. Since 10-HCPT led to an accumulation of cells in the G2/M phase as demonstrated in Example 3, we speculated that the underlying molecular mechanism might involve changes in the expression of cell cycle regulatory proteins. To confirm this speculation, we analyzed the expressions of several cell cycle regulatory proteins by means of Western blotting assay as described below.

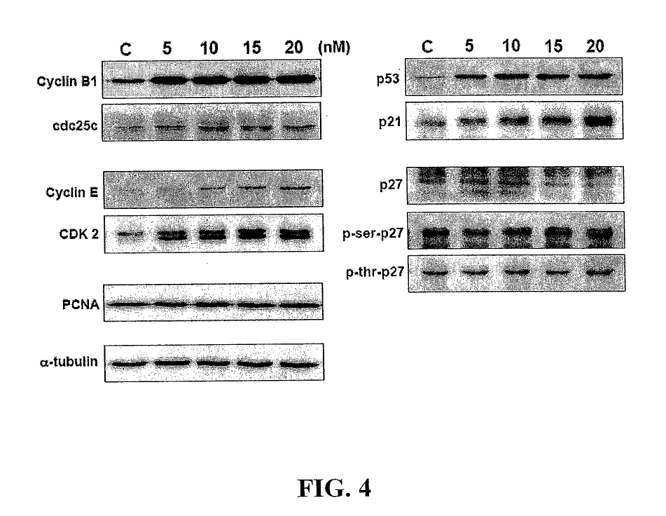

Colo-205 cells were cultured in Dulbecco's modified Eagle Medium (DMEM) supplemented with 10% fetal bovine serum (FBS) at 37° C. in a 5% CO₂ atmosphere. Cells were seeded in 6-well plate and incubated with 3 μM of 10-HCPT. After 48 hr, the treated cells were washed twice with phosphate-buffered saline (PBS), flash frozen in liquid nitrogen, and stored at −80° C. The harvested cells were lysed in ice-cold report lysis buffer (Promega). After cleaning the resulted lysates by centrifugation, protein in the clear lysate was quantified by Dc protein assay kit (Bio-Rad). 15 μg of lysate protein were resolved by 10% SDS-PAGE, transferred onto a polyvinylidene difluoride membrane (PVDF membrane, Amersham Biosciences), and immunoblotted with antibodies against followed cellular proteins: DNA topoisomerase I, p53, p27, phosphorylated p27 (Santa Cruz Biotechnology), cyclin E, CDK2 (BD Pharningen), PCNA, p21 (Upstate), and α-tubulin (Sigma). Protein content was visualized with BM Chemiluminescence Blotting Kit (Roche Molecular Biochemicals). The blots were exposed to X-ray film (Fuji) for various times. The results are shown in FIG. 4.

As can be seen from FIG. 4, the increasing expression of cyclin E and CDK2 and the decreasing amount of p27 indicated that 10-HCPT did not interfere cells bypassing the G1/S checkpoint and entering the S phase. p21 protein level dramatically increased in a 10-HCPT dose-dependent manner. It resulted in a strong G2/M arrest of cell cycle, although the expressions of both cyclin B1 and cdc25c enhanced by 10-HCPT treatments. In contrast, 10-HCPT showed no effect on PCNA protein level. Thus, the dose-dependent expressional changes of CDK2, cyclin E, cyclin B1, p21, and p27 corresponded with the 10-HCPT-induced effect on cell cycle progression (FIGS. 3 and 4).

### EXAMPLE 5

### Effect of 10-HCPT in Inducing Apoptosis in Colo-205 Cell Line

Because apoptosis always takes place as a consequence of a cell cycle block, it is possible that 10-HCPT is able to cause cell death through apoptosis pathway. To verify this possibility, we first performed DNA fragmentation assay since DNA fragmentation is one characteristic of apoptosis.

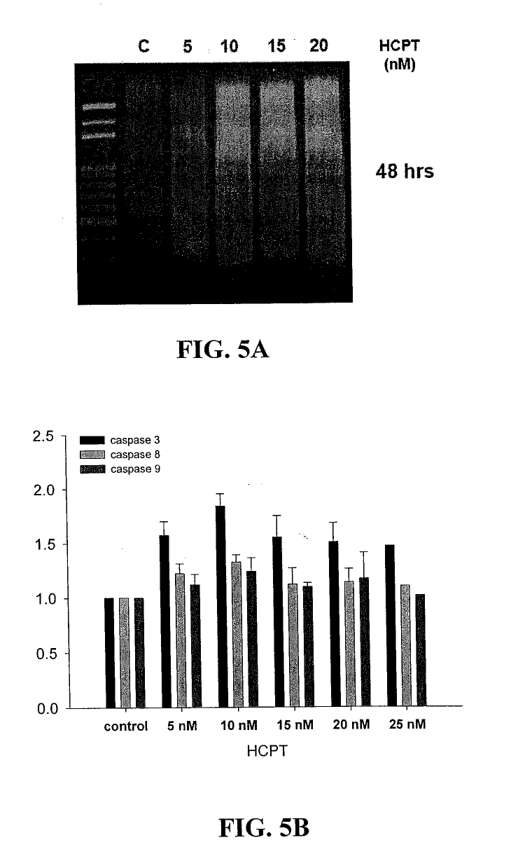

The DNA of Colo-205 cells treated with various concentrations of 10-HCPT for 48 hrs was extracted and analyzed by 1.5% native agarose gel electrophoresis. As shown in FIG. 5A, untreated cells showed no DNA ladders whereas the DNA ladders were detected in 10-HCPT-treated cells. Increasing the concentration of 10-HCPT resulted in the enhancement of DNA ladder. This result, consistent with our previous data shown in FIG. 3, suggests that 10-HCPT is capable of inducing apoptosis in Colo-205 cells.

In addition, to further determine whether caspases participate in the propagation of apoptosis induced by 10-HCPT in Colo-205 cells, cell lysates from drug-treated cells were analyzed for the cleavage of the p-nitroaniline-tagged peptides N-Acetyl-Asp-Glu-Val-Aps-pNA (DEVD-pNA), which represent preferential substrates for caspase-3. Caspase activation leads to the release of pNA that can be monitored by absorbance measurements at 405 nm using a plate reader. Specifically, DEVD-pNA cleavage activity was measured using Caspase-3 Colorimetric Assay Kit (BioVision) according to the recommended protocols. 3×10⁶ of 10-HCPT treated cells were harvested, resuspended in 50 μl of chilled cell lysis buffer and incubated on ice for 10 min before centrifugation (12,000 rpm for 3 min at 4° C.). The supernatant was collected and the total protein concentration of the supernatant was determined by Dc protein assay kit (Bio-Rad). Each assay reaction containing 100 μg of cell lysates, 50 μl of 2× reaction buffer supplemented with 10 mM DTT, and 5 μl of the 4 mM of DEVD-pNA (200 μM final concentration) was carried out at 37° C. for 1 hr. The formation of p-nitroanilide was detected at 405 nm in a microtiter plate reader.

With the DEVD-pNA substrate, the absorbance measured after 10-HCPT treatment of the cells for 48 hrs was clearly elevated compared to that measured in the control (drug-free) lysates (FIG. 5B). In contrast, with both IETD-pNA and LEHD-pNA substrates, 10-HCPT only stimulated little caspase activations in Colo-205 cells. These results imply that apoptosis induced by 10-HCPT is through the caspase-3 dependent pathway.

### EXAMPLE 6

### In Vivo Antiproliferation Effect of 10-HCPT for Human Colon Cancer Cells Colo-205 Xenografts

We further examined whether 10-HCPT is effective in growth suppression in vivo after tumor formation. Colo-205 cells were transplanted into BALB/c-nu mice, and when the tumors were palpable (3-5 mm), the mice were treated either with vehicle control or 10-HCPT.

The in vivo experiment was carried out with ethical committee approval and met the standards required by the Guiding Principles in the Use of Animals in Toxicology, which were adopted by the Society of Toxicology in 1989. The BALB/c-nu mice were obtained from National Laboratory Animal Center of National Applied Research Laboratories (Taipei, Taiwan) and housed in a laminar flow room under sterilized conditions with the temperature being maintained at 25±2° C. and the light controlled at 12 h light and 12 h dark cycle. The Colo 205 cells were harvested and resuspended in serum free RPMI-1640 medium. Cells were adjusted to 1×10⁷ cells/mL, and transplanted subcutaneously on the flank regions of the mice. Each experimental group included six to seven mice bearing tumors.

To calculate the suitable dosage for mice, we adopted the scaling method. That is, we used the dose for human as reference to calculate the equivalent dose for mice. The dose for mice was calculate through the following formula:

Dose for Mice (mg/kg/day)=Dose for Human (mg/kg/day)×[65 kg (Human Average Body Weight)]/1600 (Human MEC Value)]×[3.7 (Mice MEC Value)/0.02 (Mice Average Body Weight)]

[MEC: minimum energy cost, which equals to the basal metabolism rate (BMR); for more reference, see Ruckebush, Y. et al., Physiology of Small and Large Animals, pp. 387-398. B. C. Decker Inc., USA, 1991]

10-HCPT was dissolved in propylene glycol and treatment began when the tumor size reached 3-5 mm. 10-HCPT was administered via p.o. once per two or four days at doses of 1-7.5 mg/kg (volume of injection: 0.1 mL/20 g of body weight), respectively. The control group received propylene glycol vehicle once per two days. Tumor size and body weight were monitored twice a week throughout the experiment. The tumor size was measured using a vernier caliper. Tumor volume (V) was calculated according to the formula: V (mm³)=0.4AB², where A and B are the longest diameter and the shortest diameter, respectively. At the end of experiment, all mice were sacrificed by CO₂ gas. Tumors, livers, kidneys, and lungs were collected, fixed, embedded and stained with hematoxylin and eosin for pathological analysis.

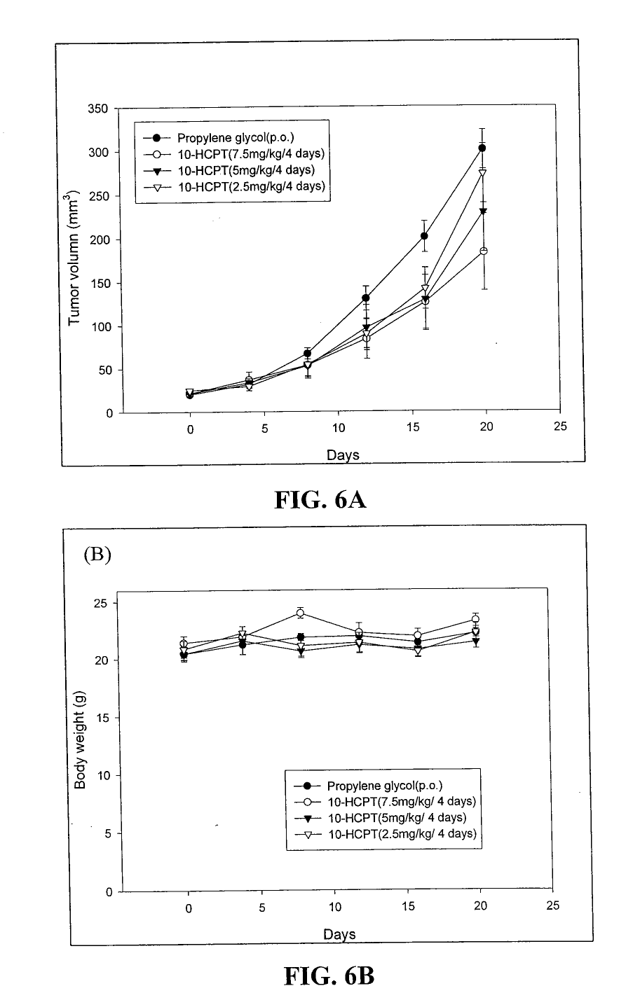

FIG. 6 shows that the tumor volume from control animals showed an average of 300.5±22.3 mm³ at the end of this study (day 24). In contrast, the tumor volume from 10-HCPT (2.5, 5 or 7.5 mg/kg/2 days)-treated animals had an average of 85.9±12.3, 60.8±15.4 and 7.2±2.8 mm³, respectively (FIG. 6A). These were significantly (P<0.01) decreased in mice as compared to control groups at the end of study (day 24). The tumor volume from 10-HCPT (2.5, 5 or 7.5 mg/kg/4 days)-treated animals at day 24 had an average of 273.1±33.7, 228.9±45.3 and 182.9±43.9 mm³, respectively (FIG. 6B). The tumor volume was significantly (p<0.05) decreased in 5 and 7.5 mg/kg/4 days-treated group as compared to control group at the end of study (day 24), although to a lesser extent in those mice treated with 10-HCPT 2.5-7.5 mg/kg/4 days.

The challenge of 10-HCPT (1-7.5 mg/kg, p.o., once per two or four days) in nude mice produced no obvious acute toxicity. No significant reduction in body weight was found in 10-HCPT-treated mice, except the 7.5 mg/kg/2 days-treated group (FIG. 6, (B)). The average body weight of 10-HCPT 7.5 mg/kg/2 days-treated group was slightly recovered at the end of study. In addition, no tissue damage was observed in liver, lung and kidney after examination of the tissue slices stained with hematoxylin and eosin (data not shown).

Previous pharmacokinetic studies indicate that 10-HCPT has a short distribution half-life and a prolonged elimination half-life. Dose-dependent toxicity was observed with i.v. administration of 10-HCPT. It can be best administered as a loading dose followed by a maintenance dose every other day. Since large amounts of drug were excreted within a short period following i.v. administration, the possible side effects on renal function may be observed, especially in the early phase of drug disposition (Zhang, R. et al., Cancer Chemother Pharmacol 41: 257-67 (1998)).

The results in Example 6 demonstrate that following oral administration at doses of 2.5-7.5 mg/kg/2 days, but not 2.5-7.5 mg/kg/4 days, tumor growth suppression by 10-HCPT was obviously observed in nude mouse xenografts. The challenge of 10-HCPT (2.5-7.5 mg/kg, p.o., once per two or four days) in mice produced no obvious acute toxicity. No significant reduction in body weight was found in 10-HCPT-treated mice, except the 7.5 mg/kg/2 days-treated group. These results agree with the studies of R. Zhang, and the low maintenance dose of 10-HCPT is the best oral administration strategy. We concluded that 10-HCPT could be administered orally and the long-term low maintenance dose of 10-HCPT should be the best administration strategy to achieve maximal anticancer effect.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/We claim:

1. An oral pharmaceutical composition for the treatment of digestive tract cancers, comprising an effective amount of a camptothecin derivative dissolved in a pharmaceutically acceptable, liquid vehicle, wherein the camptothecin derivative is administered at a dose of about 0.05 to about 1 mg/kg body weight/day.

2. The pharmaceutical composition according to claim 1, wherein the camptothecin derivative is a compound of the following formula: 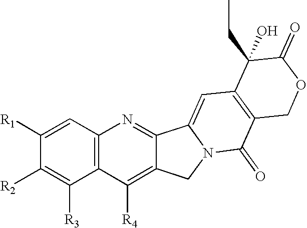
wherein R₁ represents H, —OCH₂O or —O(CH₂)₂O; R₂ represents H, OH, —OCH₂O, —O(CH₂)₂O, —OR wherein R is —C(O)—Cₙ alkyl and n is 9, 11, 13, 15, 17 or 19, or —OC(O)-piperidyl-piperidyl; R₃ represents H or —CH₂N(CH₃)₂; and R₄ represents H, methyl, ethyl, n-butyl, —Si(CH₃)₃ or —Si(CH₃)₂-t-butyl.

3. The pharmaceutical composition according to claim 2, wherein the camptothecin derivative is 10-hydroxycamptothecin (10-HCPT).

4. The pharmaceutical composition according to claim 1, wherein the liquid vehicle is a non-aqueous solvent selected from the group consisting of propylene glycol, DMSO, PEG and cremophor.

5. The pharmaceutical composition according to claim 4, wherein the non-aqueous solvent comprises propylene glycol.

6. The pharmaceutical composition according to claim 1, wherein the liquid vehicle is an aqueous vehicle with the derivative dissolved in a solubilizing agent.

7. The pharmaceutical composition according to claim 1, wherein the digestive tract cancer is colorectal cancer (CRC).

8. The pharmaceutical composition according to claim 1, wherein the camptothecin derivative is administered at a dose of about 0.15 to about 0.5 mg/kg body weight/day.

9. A method for treating digestive tract cancers, comprising administering to an animal having a digestive tract cancer an effective amount of a pharmaceutical composition comprising a camptothecin derivative dissolved in a pharmaceutically acceptable, non-aqueous solvent, wherein the camptothecin derivative is administered at a dose of about 0.05 to about 1 mg/kg body weight/day.

10. The method according to claim 9, wherein the campotothecin derivative is a compound of the following formula: 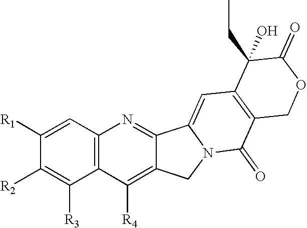
wherein R₁ represents H, —OCH₂O or —O(CH₂)₂O; R₂ represents H, OH, —OCH₂O, —O(CH₂)₂O, —OR wherein R is —C(O)—Cₙ alkyl and n is 9, 11, 13, 15, 17 or 19, or —OC(O)-piperidyl-piperidyl; R₃ represents H or —CH₂N(CH₃)₂; and R₄ represents H, methyl, ethyl, n-butyl, —Si(CH₃)₃ or —Si(CH₃)₂-t-butyl.

11. The method according to claim 10, wherein the camptothecin derivative is 10-hydroxycamptothecin (10-HCPT).

12. The method according to claim 9, wherein the non-aqueous solvent is selected from the group consisting of propylene glycol, DMSO, PEG, and cremophor.

13. The method according to claim 12, wherein the non-aqueous solvent comprises propylene glycol.

14. The method according to claim 9, wherein the digestive tract cancer is colorectal cancer (CRC).

15. The method according to claim 9, wherein the camptothecin derivative is administered at a dose of about 0.15 to about 0.5 mg/kg body weight/day.
